(19) United States
(12) Patent Application Publication
(10) Pub. No.: US 2009/0209046 A1
(43) Pub. Date: Aug. 20, 2009

(54) Neutral Pharmaceuticals

(76) Inventors: Brian Douglas Moulton, Somerville, MA (US); Zhenbo Ma, Providence, RI (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/227,471

(22) PCT Filed: May 21, 2007
(86) PCT No.: PCT/US07/11990

§ 371 Date: Mar. 30, 2009

Related U.S. Application Data
(60) Provisional application No. 60/802,461, filed on May 22, 2006.

(51) Int. Cl. G01N 33/566; C07F 19/00

(52) U.S. Cl. 436/501; 546/6; 544/225

(57) ABSTRACT

The invention comprises neutral multi-functionality assemblies of pharmaceuticals comprising an active medicinal functionality, a transition metal functionality, and an ancillary ligand functionality. An exemplary series of mixed-ligand coordination complexes comprised of copper(II), a drug and an ancillary ligand were made and tested. It is demonstrated that the judicious choice of an ancillary ligand affords a large degree of control over die relative lipophilicity/hydrophilicity of the complex in relation to the uncomplexed drug molecule. The important factors to be considered in the design of such complexes, such as the additive-constitutive nature of the partition coefficient of the ancillary ligand and the relative size of the two types of ligands are disclosed, and methods of designing neutral multi-functionality assemblies of pharmaceuticals are disclosed.

### BACKGROUND

Drug development is a complicated and risky undertaking. After much trial and error research and bench top development and scale up, a “parent” drug is created and shows some safety and efficacy in clinical trials. Additional clinical trials are conducted and the drug finally obtains market approval and enters the market. The drug works but has side effects. More research and development is undertaken, this time to modify the “parent” drug to eliminate or lessen the side effects, and possibly to improve efficacy or biological activity. Currently, methods of this “second generation” drug development include high throughput chemistry and combinatorial chemistry. In high throughput chemistry large libraries of potential drug candidates are screened for bioactivity and selectivity. When several compounds are identified that share common chemical features, pharmacophores, chemical analogs that contain the set of structural features recognized as responsible for bioactivity, are created to improve the features.

Medicinal chemistry involves the design and synthesis of molecules having a therapeutic benefit, and is most often associated with the discovery and early development stages of drug's life cycle. Furthermore, the primary focus is generally the derivitization of lead compounds or drug candidates via covalent organic transformations. Despite the rapid evolution of supramolecular synthesis¹, the concept of supramolecular medicinal chemistry² is unfamiliar to most practitioners, with the exception of its inherent application in drug design, such as in molecular docking studies. The recent emergence of pharmaceutical co-crystals³, which exploit non-covalent interactions (i.e. hydrogen bonding) for the supramolecular synthesis of multiple component crystalline materials suitable for therapeutic use, may also be regarded as supramolecular medicinal chemistry; however, thus far, it has only managed to gain industrial recognition during formulation studies. The search for safer and more efficacious drug compounds had lead to the development of, for example, copper complex NSAIDs, with enhanced anti-inflammatory activity and reduced gastrointestinal toxicity compared with the uncomplexed “parent” drug. But these apparently low toxicity drugs have yet to make a significant market impact.⁴

Improving the methods employed to solve the problems of parent drug solubility, dissolution, absorption, stabilization, complexation, encapsulation and/or toxicity will result in a more streamlined and economical drug discovery process.

### SUMMARY OF THE INVENTION

The invention is based on the concept that parent drug molecules can be complexed with transition metal cations of known desirable characteristics in chromophoric structures in order to improve their pharmocogenic properties and profiles. Rather than defining pharmacophore mimics of the parent drug's basic biological activity and testing each mimic for the biological profiles needed relating to solubility, toxicity, absorption, bioavailability, stability etc., this concept teaches the use of ligands with known behavior altering profiles to create neutral multi-functionality assemblies including the active pharmaceutical functionality. The supramolecular synthesis of mixed-ligand metal complexes that possess active pharmaceutical functionalities (APFs) as ligands⁵ offers another approach to the synthesis of pharmaceutical co-crystals for affecting key therapeutic parameters such as solubility and bioavailability.

Accordingly, in one aspect the invention comprises neutral multi-functionality assemblies of pharmaceuticals comprising an active medicinal functionality (AMF), and one or more behavior-altering functionalities, either a transition metal functionality (TMF), an ancillary ligand functionality (ALF), or both. The TMF comprises one or more transition metal cations to which are covalently bonded anionic elements including one or more active pharmaceutical functionality (APFs) and one or more ALFs such that the total molecular charge is neutral.

The transition metal cations can have a charge of +1, +2, +3, or +4. A charge of +2 or +3 is preferable. The TMFs may be “biological transition metal functionalities” (“bioTMFs”), that is, transition metal cations derived from transition metals having known low toxicity in humans. Exemplary transitions metals that can be employed in the invention include Iron (Fe), Zinc (Zn), Copper (Cu), Manganese (Mn), Chromium (Cr), Molybdenum (Mo), Cobalt (Co), Nickel (Ni), Vanadium (V), Silver (Ag), Platinum (Pt), Gold (Au), Scandium (Sc), Titanium (Ti), Yttrium (Y), Zirconium (Zr), Niobium (Nb), Technetium (Tc), Ruthenium (Ru), Rhodium (Rh), palladium (Pd), Cadmium (Cd), Hafnium (Hf), Tantalum (Ta), Tungsten (W), Rhenium (Re), Osmium (Os), Iridium (Ir), Mercury (Hg), and the inner transition metal members of the lanthanide and actinide series [(La) and (Ac) members]. Those employable as bioTMFs include Ma, Fe, Zn, and Cu. Cu is especially preferred.

The active pharmaceutical functionality, APF, may be any pharmaceutical molecule or ion containing one or more moieties to which the other functionalities may bind. Exemplary moieties include acids, amides, aliphatic nitrogen bases, unsaturated aromatic nitrogen bases such as pyridines and imidazoles, amines, alcohols, halogens, sulfones, nitro groups, S-heterocycles, saturated or unsaturated N-heterocycles, O-heterocycles, ethers thioethers, thiols, esters thioesters, thioketones, epoxides, acetonates, nitriles, oximes, and organohalides.

The ancillary ligand functionality, ALF, may be a solvent, another pharmaceutical molecule, a GRAS compound (“Generally Regarded As Safe”, by the United States Food and Drug Administration), or an approved food additive. Either the APF or the ALF must be anionic such that the total charge on the multi-functionality assembly is neutral.

In another aspect, the invention includes a method of designing a neutral multi-functionality assembly of an active pharmaceutical composition composed of an active medicinal functionality, a cationic transition metal functionality, and an ancillary ligand functionality. In this aspect, the method comprises selecting an active medicinal functionality having a moiety capable of covalently binding a cationic transition metal functionality, identifying an ancillary ligand functionality have a moiety capable of covalently binding the cationic transition metal functionality, and arranging the functionalities such that the total molecular charge of the assembly composted of the combined functionalities is neutral. In a preferred embodiment of the method, the TMF is a bioTMF, most preferably, the bioTMF is Cu. The ancillary ligand functionality is a solvent, another pharmaceutical molecule, or a GRAS compound; and the active medicinal functionality contains one or more moieties selected from the group consisting of acids, amides, aliphatic nitrogen bases, unsaturated aromatic nitrogen bases such as pyridines and imidazoles, amines, alcohols, halogens sulfones, nitro groups, S-heterocycles, saturated or unsaturated N-heterocycles, O-heterocycles, ethers thioethers, thiols, esters, thioesters, thioketones, epoxides, acetonates, nitriles, oximes, and organohalides. Either the APF or the ALF must be anionic such that the total charge on the multi-functionality assembly is neutral.

### DESCRIPTION OF THE DRAWINGS

FIG. 1(a), (b), (c) are schematic illustrations of the coordination chromophores of the Cu(II) complexes. (a) 5-coordinate paddle wheel; (b) 5-coordinate square pyramidal; (c) 4-coordinate square planar.

FIG. 2 is a reproduction of the ESI-MS for ASP-5 in water.

FIG. 3 is a reproduction of the ESI-MS for ASP-5 in octanol saturated water.

FIG. 4 is a reproduction of the ¹H NMR spectra of ASP-3 and ASP-3/3-Br-Py mixture in D₂O.

FIG. 5 is a reproduction of the ¹H DOSY spectrum of ASP-3 in D₂O.

FIG. 6 is a graphic representation of the calculated logP values for ancillary ligands (dark gray); Observed logP values for aspirin and Cu-ASP-AL complexes (light gray); Observed logSR values for aspirin and Cu-ASP-AL complexes (black).

FIG. 7 is a graphic representation of the calculated logP values for ancillary ligands (dark gray); Observed logP values for salsalate and Cu-SAS-AL complexes (light gray); Observed logSR values fox salsalate and Cu-SAS-AL complexes (black).

FIG. 8(a), (b), (c), (d), and (e) are illustrations of the single crystal structure unit of the complexes SAS-2, SAS-3, SAS-6, SAS-4 and SAS-5 in CPK mode, respectively, with the ancillary ligand functionalities shown in the solid, light gray shade.

FIG. 9 is a graphic representation of the observed logP and logSR values for complexes MC-1 through MC-5.

FIG. 10 is a graphic representation of the calculated logP values for the conjugated acid of ancillary ligand functionalities (dark gray); Observed logP values for caffeine and Cu-CAF-AL complexes (light gray), the logP was multiplied by 10 on the graph for clarity; Observed logSR values for caffeine and Cu-CAF-AL complexes (black).

FIG. 11(a)-(d) are graphic reproductions of the powder XRD patterns of complexes SAS-1 through SAS-9, MC-2 through MC-5, and CAF-1 through CAF-5.

FIG. 12 is an illustration of the chemical structures of the 28 exemplary supramolecular complexes of the invention that were made and tested in the example. ASP: aspirin and copper (II)-aspirinate complexes; SAS: salsalate and copper (II) salsalate complexes; MC: missed carboxylate copper (II) complexes; CAF: caffeine and copper (II) carboxylate-caffeine complexes.

### DETAILED DESCRIPTION

### 1. Definitions and Abbreviations

The terms “pharmaceutical(s)” and drug(s)” refer to any biologically active compound capable of having a therapeutic effect on a mammal with a pathological disease or condition. The therapeutic effect may be palliative, curative, or prophylactic. The terms include pharmaceutically acceptable salts.

“Pharmaceutically acceptable salts” means salts prepared from pharmaceutically acceptable, non-toxic, acids or bases, including inorganic acids and bases and organic acids and bases. Suitable pharmaceutically acceptable base addition salts include, for example, metallic salts made from aluminum, calcium, lithium, magnesium, potassium, sodium, or organic salts made from chloroprocaine, choline, diethanolamine, ethylenediamine, lysine, N,N′-dibenzylethylenediamine, N-methylglucamine, or procaine. Suitable exemplary non-toxic acids include, for example, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isothionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric, or p-tolenesulfonic acids. Specific, preferred, non toxic acids include hydrochloric, hydrobromic, methanesulfonic, phosphoric, or sulfuric acids. Specific salts include hydrochloride or mesylate salts. Other examples of pharmaceutically acceptable slats are well known to those skilled in the art, see, e.g., Remingon's Pharmaceutical Sciences, 18^{th} ed., Mack Publishing, Easton Pa. (1990).

The term “behavior altering functionality” refers to and includes a transition metal functionality (TMF), an ancillary ligand functionality (ALF), or both. The TMF comprises one or more transition metal cations to which are covalently bonded anionic elements including one or more active pharmaceutical functionalities (APFs) and one or more ancillary ligand functionalities (ALFs), such that the total molecular charge is neutral.

ASP: aspirin (acetylsalicylic acid, 2-(acetyloxy)-benzoid acid), and copper(II)-aspirinate complexes

SAS: salsalate (salicylsalicylicacid, 2-hydroxybenzoicacid-2-carboxyphenylester), and copper(II)-salsalate complexes

MC: mixed carboxylate copper(II) complexes

CAF: caffeine and copper(II) carboxylate-caffeine complexes

DMF: N,N-dimethylformamide

THF: tetrahydrofuran

Py: pyridine

Me: methyl

Et: ethyl

Ph: phenyl

API: APF, active pharmaceutical functionality

AL: ancillary ligand functionality

QSAR: quantitative structure-activity relationships;

SR: solubility ratio (S_{wo}/S_{ow})

AcCN: 2-oxo-propanenitrile, Acetyl cyanide

w: water, H₂O

wo: water saturated octanol

ow: octanol saturated water

NSAIDs: non-steroidal anti-inflammatory drugs

ADMET: Absorption, Distribution, Metabolism, Excretion, and Toxicity

### 2. Description

In the context of transition metal complexes, there are two classifications of ligands: neutral and charge-compensating (i.e. anionic). There are, therefore, three primary classes of mixed-ligand pharmaceutical coordination species: anionic APF with neutral ancillary ligand functionality, anionic ancillary ligand functionality with neutral APF, and species with both anionic APF and ancillary ligand functionality (the latter may, or may not, have additional neutral ancillary ligands, such as solvent molecules). Herein we describe the synthesis and characterization of examples of all three classes of complexes and report their bulk solubility in water and octanol, solubility ratio and partition coefficient.

Although the invention is illustrated by means of the following examples using Cu as the TMF (in this case the bioTMF), aspirin, salsalate, as the AMF, and as the ALF, quinoline, water, pyridines (for example methyl, ethyl or phenyl pyridine), caffeine, nicotinamide, isonicotinamide, and various other ancillary ligand functionalities, it is understood that any transition metal may be employed instead of Cu as the TMF, and that the ALFs employed may be selected from others meeting the necessary criteria as discussed above. It is also understood that aspirin or salsalate are merely exemplary AMFs, which can be replaced with any other pharmaceutical compound. The examples illustrate the use of TMFs in drug development. With the existence of a transition metal in the pharmaceutical compound, the intrinsic properties of the metal, such as a coordination mode, can also affect the physical properties of the pharmaceutical. The pharmacodynamic properties can thus be fine-tuned with the employment of these TMF-APF-ALF systems.

### 3. Examples

### A. Materials

1-octanol (ACS reagent, >990%) were ordered from Aldrich Chemical Co. Deionized water with resistivity up to 18.3 MΩ/cm were prepared by a NANOpure Ultrapure Water System. All other materials were obtained from Aldrich Chemical Co. or VWR International Inc. and used as received.

### B. Methods

Single crystal X-ray diffraction data were collected on a BRUKER SMART-APEX CCD diffractometer using Mo_{Kα} radiation (λ=0.71073 Å). Thermogravimetric analysis was performed under nitrogen on TA instruments TGA Q500 Hi-Res. XRPD data were collected on a Bruker D8 Diffractometer at 40 KV, 40 mA for Cu_{Kα}(λ=1.5418 Å), with a step size 0.01° in 2θ at room temperature. UV-Vis spectra were recorded on a HP 8452A DIODE ARRAY UV-Vis spectrometer. ESI (Electrospray Ionization)-MS spectra were recorded on an Applied Biosystem QSTAR instrument. IR spectra were recorded on a ATI Mattson Infinity series FTIR instrument. NMR spectra were obtained on Bruker Avance 400 spectrometer.

NMR tubes with a 5-mm internal diameter were used. The diffusion NMR experiment was performed at 298K with a Stimulated Echo Sequence (STE) using bipolar gradient pulse pair. Diffusion coefficients were measured by incrementing the amplitude of the field gradient pulse over 32 steps (0.5-30 G/cm). The bipolar gradient duration and the diffusion time were optimized to 2.4 ms and 14.9 ms, respectively. The intensities were fitted to an exponential decay using the SimFit program within the Topspin software to provide estimates of the diffusion constant.

### C. Measurement of P

Partition coefficients of complexes were determined in 1-octanol/water system. 1-octanol and water were mutually saturated before use. The octanol saturated water (ow) layer was used to prepare the stock solution, generally 25 ml of which was stirred vigorously, in triplicate, with 50 ml of water saturated octanol (wo) at 25□ overnight. The organic layer was collected, and centrifuged at 7000 rpm for 20 min to get rid of trace amount of water. The organic layer was analyzed by UV. The partition coefficient was determined from the Equation:

$$P = \frac{{C}_{wo}}{( m - {C}_{wo} {V}_{wo} ) / {V}_{ow}}$$

where, m represents the total mass of the sample; C_{wo} represents equilibrium solute concentrations of the octanol phase; V_{ow} and V_{wo} represent the volume of aqueous and octanol phases, respectively.

The only exception is the measurement of P of copper acetate monohydrate (Cu₂(CH₃COO)₄(H₂O)₂). The octanol saturated layer were collected. Then extra amount of NH₄OH solution were added and mixed well. The mixture were analyzed by UV-Vis at 615 nm. The partition coefficient was determined from the Equation:

$$P = \frac{( m - {C}_{ow} {V}_{ow} ) / {V}_{wo}}{{C}_{ow}}$$

### D. Measurement of Solubility

Solubilities were determined at 25° C. in water (S_{w}), octanol saturated water (S_{ow}), water saturated octanol (S_{wo}). An excess of the sample was added to 15 ml of each solvent and stirred vigorously at 25° C. for 8 hours. This was then ported immediately into centrifuge tubes, and centrifuged at 7000 rpm for 20 min. The supernatant was collected. The supernatant samples after appropriate dilutions with respective solvent were analyzed by UV spec using a standard plot of solute in the same medium. Solubility of copper acetate in water saturated octanol was determined by extracting 200 ml saturated wo solution into 20 ml ow. Then the aqueous (ow) layer were analyzed by UV-Vis at 615 nm after adding proper amount of NH₄OH solution.

### E. Example 1

### Synthesis and Characterization of Mixed Ligand Complexes

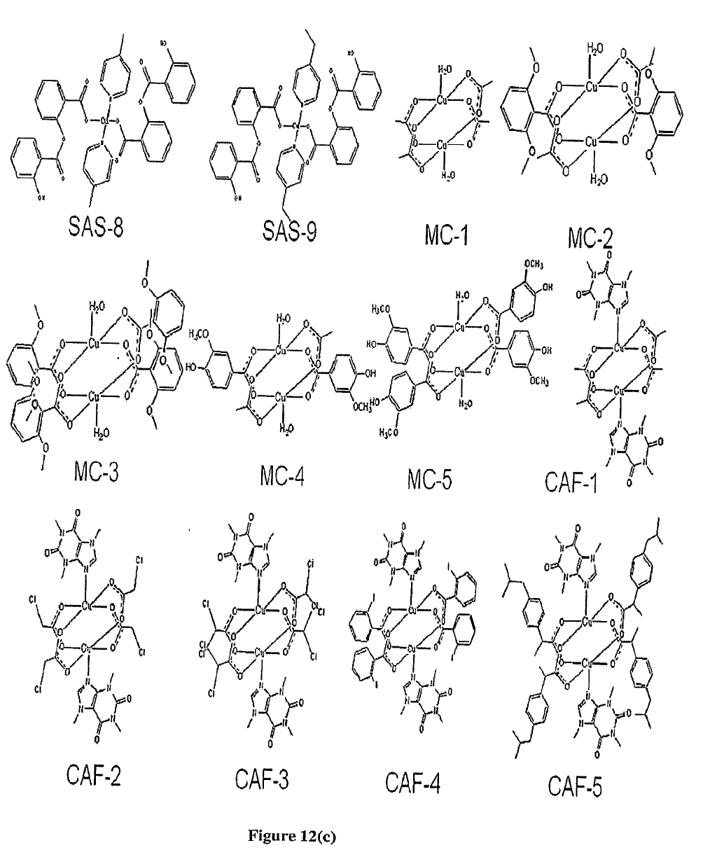

FIG. 12 illustrates the structures of the 28 exemplary complexes made and analyzed. The formulae for the complexes are set forth in Table 1, below.

TABLE 1
Formula and Coordination Chromophore of 28 Exemplary Cu(II) Complexes

| Complex | | | |
|---|---|---|---|
| No | Ancillary Ligand Functionality | Complex Formula | Coordination Chromophore |
| ASP-1 | | Cu₂(ASP)₄ | 4 coordinate paddle-wheel |
| ASP-2 | DMF | Cu₂(ASP)₄(DMF)₂ | 5 coordinate paddle-wheel |
| ASP-3 | 3-bromopyridine | Cu₂(ASP)₄(3-Br-Py)₂ | 5 coordinate paddle-wheel |
| ASP-4 | Quinoline | Cu₂(ASP)₄(Quinoline)₂ | 5 coordinate paddle-wheel |
| ASP-5 | Pyridine | Cu(ASP)₂(Pyridine)₂ | 4 coordinate square planar |
| ASP-6 | Isonicotinamide | Cu(ASP)₂(Isonicotinamide)₂(AcCN)₂ | 4 coordinate square planar |
| ASP-7 | Isonicotinamide | Cu(ASP)₂(Isonicotinamide)₂ | 4 coordinate square planar |
| ASP-8 | Nicotinamide | Cu(ASP)₂(Nicotinamide)₂ | 4 coordinate square planar |
| ASP-9 | 3-phenylpyridine | Cu(ASP)₂(3-Phenyl-Pyridine)₂ | 4 coordinate square planar |
| SAS-1 | | Cu₂(SAS)₄(H₂O)₂ | 5 coordinate paddle-wheel |
| SAS-2 | Caffeine | Cu₂(SAS)₄(caffeine)₂ | 5 coordinate paddle-wheel |
| SAS-3 | 3-chloropyridine | Cu₂(SAS)₄(3-Cl-Pyridine)₂ | 5 coordinate paddle-wheel |
| SAS-4 | 4-benzylpyridine | Cu₂(SAS)4(4-Benzyl-Pyridine)₂ | 5 coordinate paddle-wheel |
| SAS-5 | 4-phenylpyridine | Cu₂(SAS)₄(4-Phenyl-Pyridine)₂(THF)₂ | 5 coordinate paddle-wheel |
| SAS-6 | Pyridine | Cu(SAS)₂(Pyridine)₃ | 5 coordinate square pyramidal |
| SAS-7 | Isonicotinamide | Cu(SAS)₂(Isonicotinamide)₂(AcCN)_{2/3} | 4 coordinate square planar |
| SAS-8 | 4-methylpyridine | Cu(SAS)₂(4-Me-Pyridine)₂ | 4 coordinate square planar |
| SAS-9 | 4-ethylpyridine | Cu(SAS)₂(4-Et-Pyridine)₂ | 4 coordinate square planar |
| MC-1 | Anionic Mixed Carboxylate | Cu₂(CH₃COO)₄(H₂O)₂ | 5 coordinate paddle-wheel |
| MC-2 | Anionic Mixed Carboxylate | Cu₂(CH₃COO)₂(DMB)₂(H₂O)₂ | 5 coordinate paddle-wheel |
| MC-3 | | Cu₂(DMB)₄(H₂O)₂ | 5 coordinate paddle-wheel |
| MC-4 | Anionic Mixed Carboxylate | Cu₂(CH₃COO)₂(VA)₂(H₂O)₂ | 5 coordinate paddle-wheel |
| MC-5 | | Cu₂(VA)₄(H₂O)₂ | 5 coordinate paddle-wheel |
| CAF-1 | Anionic Carboxylate-caffeine | Cu₂(CH₃COO)₄(Caffeine)₂ | 5 coordinate paddle-wheel |
| CAF-2 | Anionic Mixed Carboxylate- | Cu₂(ClCH₂COO)₄(Caffeine)₂ | 5 coordinate paddle-wheel |
| | caffeine | | |
| CAF-3 | Anionic Mixed Carboxylate- | Cu₂(Cl₂CHCOO)₄(Caffeine)₂ | 5 coordinate paddle-wheel |
| | caffeine | | |
| CAF-4 | | Cu₂(2-I-benzoato)₄(Caffeine)₂ | 5 coordinate paddle-wheel |
| CAF-5 | | Cu₂(Ibuprofen)₄(Caffeine)₂ | 5 coordinate paddle-wheel |

Experimental procedures employed in preparing ASP-1 and ASP-2 are detailed in Viossat et al., J. Med. Chem. 19: 135-45 (1976). The structures were characterized by single-crystal X-Ray diffraction (XRD). ASP-1 is a multi-crystalline powder and was characterized by TGA. Hi-Res Thermogravimetric analysis (TGA) resulted in peaks at 222.05° C., 226.95° C., 268.08° C., 361.93° C., weight loss 73.66%. Crystal data for ASP-2: C₄₂H₄₂Cu₂N₂O₁₈. M=989.54, Monoclinic, P-2₁/n, a=12.259(1), b=10.228(1), c=16.987(1) Å, β=92.068(9), V=2128.506 Å3, Z=2, ρ_{cal}=1.54 g/cm³, T=180(2) K, R₁(I>2σ(I))=0.030, wR₂=0.033. Deposited in CSD, ref code: OKEZUZ. The structure consists of two Cu atoms linked by four aspirin carboxylate moieties forming a paddle-wheel structure, with two DMF molecules coordinating to the Cu atoms along the Cu—Cu axis.

ASP-3 was prepared by adding 3-Br-Py to 0.5 g Cu₂(ASP)₄ until the solid was merged by 3-Br-Py in a 4 Dram glass vial. The mixture was placed in water bath at 60° C. for 1 hr and then set aside. After 1 day, the green crystal was formed. The structure was characterized by single-crystal XRD. Crystal data: C₉₂H₇₂Br₄Cu₄N₄O₃₂. M=2319.34, Triclinic, P-1, a=10.796(3), b=13.313(4), c=17.371(5) Å, α=74.133(6), β=73.352(6), γ=77.566(6), V=2275.5(12) Å3, Z=1, ρ_{cal}=1.693 g/cm³, T=90(2) K, R₁(I>2σ(I))=0.1109, wR₂=0.2729. The structure consists of two Cu atoms linked by four aspirin carboxylate moieties forming a paddle-wheel, with two 3-Br-Py molecules coordinating to Cu atoms along the Cu—Cu axis.

ASP-4 was prepared by adding quinoline to 0.5 g Cu₂(ASP)₄ until the solid was merged by quinoline in a 4 Dram glass vial. The mixture was placed in water bath at 60° C. for 1 hr and then set aside. After 1 day, the green crystal was formed. The structure was characterized by single-crystal XRD. Crystal data: C₅₄H₄₂Cu₂N₂O₁₆. M=1101.98, Triclinic, P-1, a=10.878(9), b=11.245(9), c=12.165(10) Å, α=74.226(14), β=63.597(15), γ=68.579(14), V=1230.0(17) Å3, Z=1, ρ_{cal}=1.488 g/cm³, t=90(2) K, R₁(I>2σ(I))=0.0794, wR₂=0.1420. The structure consists of two Cu atoms linked by four aspirin carboxylate moieties forming a paddle-wheel structure, with two quinoline molecules coordinating to Cu atoms along the Cu—Cu axis.

ASP-5 was prepared as described in J R J Sorenson et al., Inorg. Chim. Acta 93: 67 (1984). The structure was characterized by single-crystal XRD. Crystal data: CuC₂₈H₂₄O₈N₂. M=580.05, Monoclinic, P-2₁/n, a=17.82(1), b=10.903(7), c=6.598(4) Å, β=95.74(5), V=1276 Å³, Z=2, ρ_{cal}=1.510 g/cm³, R₁(I>2σ(I))=0.046, wR₂=0.046. Deposited in CSD, ref code CUKPED. The structure is mononuclear, with the Cu atom surrounded by four ligands in a trans square planar arrangement with two Cu—O bonds and two Cu—N bonds.

ASP-6 was prepared by taking an AcCN solution of Isonicotinamide (10 ml, 0.1 molL⁻¹), adding it to a 8 Dram glass vial. 100 mg of Cu₂(ASP)₄ was stirred vigorously and suspending in 20 ml AcCN. The suspension was carefully added into the vial. Purple crystals of Cu(ASP)₂(isonicotinamide)₂.2AcCN formed within a week under ambient conditions. The structure was characterized by single-crystal XRD. Crystal data: C₃₄H₃₂CuN₆O₁₀. M=748.20, Triclinic, P-1, a=7.7795(16), b=10.451(2), c=12.500(3) Å, α=110.06(3), β=107.97(3), γ=90.97(3), V=899.4(3) Å3, Z=1, ρ_{cal}=1.381 g/cm³, T=293(2) K, R₁(I>2σ(I))=0.0590, wR₂=0.1321. The structure is mononuclear, with the Cu atom surrounded by four ligands in a trans square planar arrangement with two Cu—O bonds and two Cu—N bonds.

ASP-7 was prepared by heating ASP-6 in oven at 100° C. overnight. Hi-Res Thermogravimetric analysis (TGA) resulted in peaks at 161.83° C., 249.60° C., weight loss 84.50%.

ASP-8 was prepared by taking an AcCN solution of Nicotinamide (10 ml, 0.1 molL⁻¹), adding it to a 8 Dram glass vial. 100 mg of Cu₂(ASP)₄ was stirred vigorously and suspending in 20 ml AcCN. The suspension was carefully added into the vial. Purple crystals of Cu(ASP)₂(isonicotinamide)₂.2AcCN formed within a week under ambient conditions. The structure was characterized by single-crystal XRD. Crystal data: C₃₀H₂₆CuN₄O₁₀. M=666.09, Monoclinic, P-2₁/n, a=8.799(2), b=9.619(3), c=17.334(5) Å, β=95.245(6), V=1461.0(7) Å3, Z=2, ρ_{cal}=1.514 g/cm³, T=293(2) KC, R₁(I>2σ(I))=0.0553, wR₂=0.1023. The structure is mononuclear, with the Cu atom surrounded by four ligands in a trans square planar arrangement with two Cu—O bonds and two Cu—N bonds.

ASP-9 was prepared by adding 3-Ph-Py to 0.5 g Cu₂(ASP)₄ until the solid was merged by 3-Ph-Py in a 4 Dram glass vial. The mixture was placed in water bath at 60° C. for 1 hr and then set aside. After 1 day, the purple crystal was formed. The structure was characterized by single-crystal XRD. Crystal data: C₄₀H₃₂CuN₂O₈. M=732.22, Triclinic, P-1, a=8.5173(16), b=8.7980(17), c=11.913(2) Å, α=101.907(4), β=98.371(3), γ=98.140(4), V=850.5(3) Å3, Z=1, ρ_{cal}=1.430 g/cm³, T=90(2) K, R₁(I>2σ(I))=0.0587, wR₂=0.1211. The structure is mononuclear, with the Cu atom surrounded by four ligands in a trans square planar arrangement with two Cu—O bonds and two Cu—N bonds.

SAS-1 was prepared following the procedures detailed in A E Underhill, J. Inorg. Biochem 37: 1 (1989). The structure was characterized by single-crystal ED. SAS-1 is a multi-crystalline powder and was characterized Hi-Res thermogravimetric analysis (TGA), which resulted in peaks at 120.06° C., 213.38° C., 282.78° C., 374.54° C., weight loss 75.73%. The structure consists of two Cu atoms linked by four salsalate carboxylate moieties forming a paddle-wheel structure, with two water molecules coordinating to Cu atoms along the Cu—Cu axis.

SAS-2 was prepared by transferring an MeOH solution of CuCl₂ (10 ml, 0.1 molL⁻¹) to a 8 Dram glass vial. 2 mMol salsalate was dissolved in 20 ml MeOH and partly neutralized by 1 mMol sodium methoxide. The salsalate solution was carefully added into the vial. Blue crystals formed within an hour under ambient conditions. The structure was characterized by single-crystal XRD. Crystal data: C₅₉H₄₈Cu₂O₂₃. M=1252.05, Triclinic, P-1, a=10.061(3), b=10.899(3), c=14.406(4) Å, α=104.463(4), β=91.665(4), γ=0.0958. Hi-Res thermogravimetric analysis (TGA) resulted in peaks at 214.27° C., 269.77° C., weight loss 48.53%. The structure consists of two Cu atoms linked by four salsalate carboxylate moieties forming a paddle-wheel structure, with two water molecules coordinating to Cu atoms along the Cu—Cu axis.

SAS-3 was prepared by slow evaporation of saturated Cu₂(SAS)₄(water)₂THF solution overnight. The green crystal was formed. The structure was characterized by single-crystal XRD. Crystal data: C₆₄H₅₂Cu₂O₂₂, M=1300.14, Triclinic, P-1, a=10.1423(16), b=10.9246(17), c=14.398(2) Å, α=104.006(2), β=94.966(2), γ=107.953(2), V=1449.9(4) Å³, Z=1, ρ_{cal}=1.489 g/cm³, T=100(2) K, R₁(I>2σ(I))=0.0529, wR₂=0.0972. Hi-Res thermogravimetric analysis (TGA) resulted in peaks at 172.79° C., 199.14° C., 279.59° C., weight loss 81.86%. The structure consists of two Cu atoms linked by four salsalate carboxylate moieties forming a paddle-wheel structure, with two THF molecules coordinating to Cu atoms along the Cu—Cu axis.

SAS-4 was prepared by adding 3-Ph-Py to 0.5 g Cu₂(SAS)₄(water)₂ until the solid was merged by 3-Ph-Py in a 4 Dram glass vial. The mixture was placed in water bath at 60° C. for 1 hr and then set aside. After 1 day, the green crystal was formed. The structure was characterized by single-crystal XRD. Crystal data: C₆₆H₄₄Cl₂Cu₂N₂O₂₀. M=1383.01, Triclinic, P-1, a=10.423(18), b=11.032(18), c=14.32(2) Å, α=104.03(3), β=94.41(3), γ=108.91(4), V=1490(4) Å³, Z=1, ρ_{cal}=1.542 g/cm³, T=90(2) K, R₁(I>2σ(I))=0.1243, wR₂=0.1648. Hi-Res thermogravimetric analysis (TGA) resulted in peaks at 186.25° C., 215.66° C., 265.90° C., weight loss 85.37%. The structure consists of two Cu atoms linked by four salsalate carboxylate moieties forming a paddle-wheel structure, with two 3-Cl-Py molecules coordinating to Cu atoms along the Cu—Cu axis.

SAS-5 was prepared by adding 0.5 g Cu₂(SAS)₄(water)₂ and 0.163 g caffeine into 10 ml AcCN in a 4 Dram glass vial. The mixture was placed in water bath at 60° C. for 1 hr and then set aside. After 1 day, the green crystal was formed. The structure was characterized by single-crystal XRD. Crystal data: C₇₂H₅₆Cu₂N₈O₂₄, M=1544.33, Monoclinic, P-2₁/n, a=12.904(2), b=17.095(3), c=15.923(3) Å, β=109.361(4), V=3313.9(11) Å³, Z=2, ρ_{cal}=1.548 g/cm³, T=90(2) K, R₁(I>2σ(I))=0.0666, wR₂=0.1060. Hi-Res thermogravimetric analysis VGA) resulted in peaks at 126.08° C., 209.15° C., 299.94° C., weight loss 76.94%. The structure consists of two Cu atoms linked by four salsalate carboxylate moieties forming a paddle-wheel structure, with two 3-Cl-Py molecules coordinating to Cu atoms along the Cu—Cu axis.

SAS-6 was prepared by adding Py to 0.5 g Cu₂(SAS)₄(water)₂ until the solid was merged by Py into a 4 Dram glass vial. The mixture was placed in water bath at 60° C. for 1 hr and then set aside. After 1 day, the blue crystal was formed. The structure was characterized by single-crystal XRD. Crystal data: C₄₃H₃₃CuN₃O₁₀. M=815.26, Monoclinic, C2/c, a=23.694(3), b=9.3561(12), c=16.664(2) Å, β=97.587(3), V=3661.8(8) Å³, Z=4, ρ_{cal}=1.479 g/cm³, T=90(2) K, R₁(I>2σ(I))=0.0519, wR₂=0.0811. Hi-Res thermogravimetric analysis (TGA) resulted in peaks at 98.73° C., 202.39° C., 306.63° C., weight loss 85.35%. The structure is mononuclear, with the Cu atom surrounded by five ligands in a square pyramidal arrangement with two Cu—O bonds and three Cu—N bonds.

SAS-7 was prepared by suspending 0.4 mg Cu₂(SAS)₄(water)₂ in 10 ml THF with vigorous stirring. The suspension was then transferred to a 8 Dram glass vial. An AcCN solution of Isonicotinamide (20 ml, 0.1 molL⁻¹) was carefully added into the vial. Purple crystals of (Cu(SAS)₂(isonicotinamide)₂.⅔(AcCN)) formed within a week under ambient conditions. The structure was characterized by single-crystal XRD. Crystal data: C₆₂H₄₈Cu_{1.5}N₇O₁₈. M=1274.38, Triclinic, P-1, a=12.415(3), b=12.601(3), c=20.925(5) Å, α=90.467(4), β=97.140(4), γ=118.314(3), V=2850.9(11) Å³, Z=2, ρ_{cal}=1.485 g/cm³, T=100(2) K, R₁(I>2σ(I))=0.0951, wR₂=0.1779. Hi-Res thermogravimetric analysis (TGA) resulted in peaks at 149.72° C., 185.00° C., weight loss 84.32%. The structure is mononuclear, with the Cu atom surrounded by four ligands in a trans square planar arrangement with two Cu—O bonds and two Cu—N bonds.

SAS-8 was prepared by adding 4-Me-Py to 0.5 g Cu₂(SAS)₄(water)₂ until the solid was merged by 4-Me-Py in a 4 Dram glass vial. The mixture was placed in water bath at 60° C. for 1 hr and then set aside. After 1 day, the purple crystal was formed. The structure was characterized by single-crystal XRD. Crystal data: C₄₀H₃₂CuN₂O₁₀, M=764.22, Monoclinic, P-2₁/n, a=7.8154(16), b=15.567(3), c=14.920(3) Å, β=104.52(3), V=1757.2(6) Å³, Z=2, ρ_{cal}=1.444 g/cm³, T=90(2) K, R₁(I>2σ(I))=0.0494, wR₂=0.0710. Hi-Res thermogravimetric analysis (TGA) resulted in peaks at 142.34° C., 206.39° C., 283.10° C., weight loss 85.12%. The structure is mononuclear, with the Cu atom surrounded by five ligands in a trans square planar arrangement with two Cu—O bonds and two Cu—N bonds.

SAS-9 was prepared by adding 4-Et-Py to 0.5 g Cu₂(SAS)₄(water)₂ until the solid was merged by 4-Et-Py in a 4 Dram glass vial. The mixture was placed in water bath at 60° C. for 1 hr and then set aside. After 1 day, the purple crystal was formed. The structure was characterized by single-crystal XRD. Crystal data: C₄₂H₃₆CuN₂O₁₀, M=792.27, Monoclinic, P-2₁/n, a=7.822(3), b=15.493(6), c=15.188(5) Å, β=102.096(8), V=1799.7(11) Å³, Z=2, ρ_{cat}=1.462 g/cm³, T=90(2) K, R₁(I>2σ(I))=0.0766, wR₂=0.0949. Hi-Res thermogravimetric analysis (TGA) resulted in peaks at 148.55° C., 203.68° C., 266.73° C., weight loss 88.23%. The structure is mononuclear, with the Cu atom surrounded by five ligands in a trans square planar arrangement with two Cu—O bonds and two Cu—N bonds.

MC-1 was obtained from Aldrich Chemical Co and used as received.

MC-2 through MC-5 complexes were prepared as described in Strinnaerre et al., Inorg. Chem. 24: 2297-2300 (1985) and in Kozlevcar, et al., Polyhedron 25: 1161-66 (2006). These complexes were characterized by powder x-ray diffraction (PXRD) and by thermogravimetric analysis (TGA) as follows.

MC-2 Hi-Res TGA: peak 166.26° C., 233.89° C., weight loss 76.82%.

MC-3 Hi-Res TGA: peak 132.21° C., 200.87° C., weight loss 74.65%.

MC-4 Hi-Res TGA: peak 51.72° C., 152.12° C., 217.27° C., 230.57° C., 258.49° C., 325.52° C., weight loss 64.95%

MC-5 Hi-Res TGA: peak 189.64° C., weight loss 38.92%; peak 294.52° C., weight loss 40.19%.

CAF-1 through CAF-5 complexes were prepared as described in Melnik et al., J. Inorg. Nucl. Chem. 43: 3035-38 (1981); Valach et al., J. Organomet. Chem. 622: 166-71 (20010; and in Abuhijleh et al., J. Inorg. Biochem 55: 255-62 (1994). These complexes were characterized by PXRD, by TGA, and by FT-IR as follows.

CAF-1 Hi-Res TGA: peak 206.58° C., 246.76° C., 358.51° C., weight loss 78.760%. IR (KBr): 1624 cm⁻¹ s (γcoo-(asym)); 1422 cm⁻¹ m (γcoo-(sym)).

CAF-2 Hi-Res TGA: peak 193.03° C., weight loss 71.15%. IR (KBr): 1650 cm⁻¹ s (γcoo-(asym)); 1413 cm⁻¹ m (γcoo-(sym)).

CAF-3 Hi-Res TGA: peak 184.98° C., 199.66° C., 221.81° C., weight loss 78.710%. IR (KBr): 1650 cm⁻¹ s (γcoo-(asym)); 1401 cm⁻¹ m (γcoo-(asym)).

CAF-4 Hi-Res TGA: peak 233.08° C., 305.58° C., weight loss 57.07%. IR (KBr): 1628 cm⁻¹ s (γcoo-(asym)); 1405 cm⁻¹ m (γcoo-(asym)).

CAF-5 Hi-Res TGA: peak 201.92° C., 236.21° C., 363.84° C., weight loss 88.92%. IR (KBr): 1662 cm⁻¹ s (γcoo-(asym)); 1405 cm⁻¹ m (γcoo-(asym)).

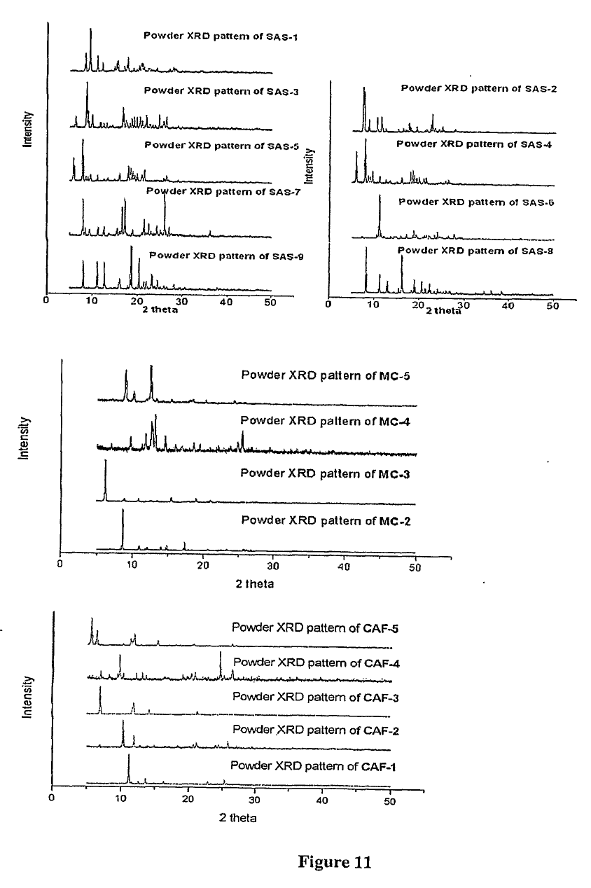

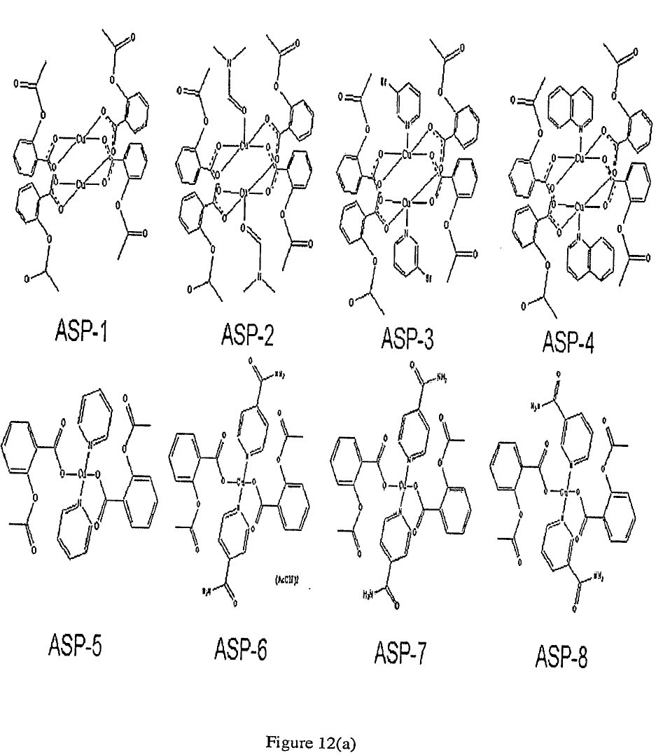

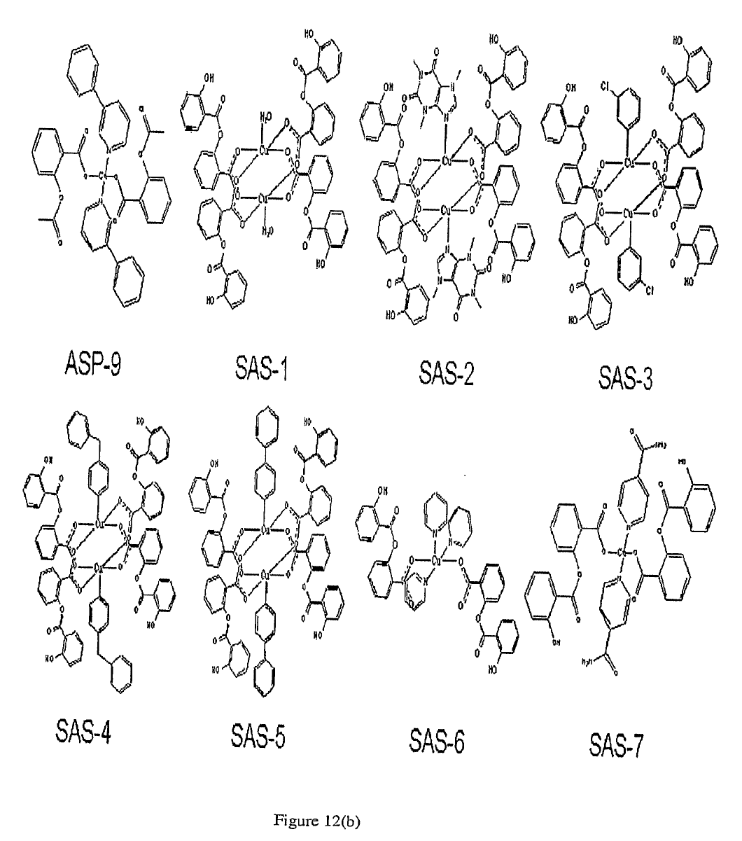

The powder XRD patterns for SAS-1 through SAS-9, MC-2 through MC-5 and CAF-1 through CAF-5 are shown in FIG. 11.

### Example 2

### Results and Discussion

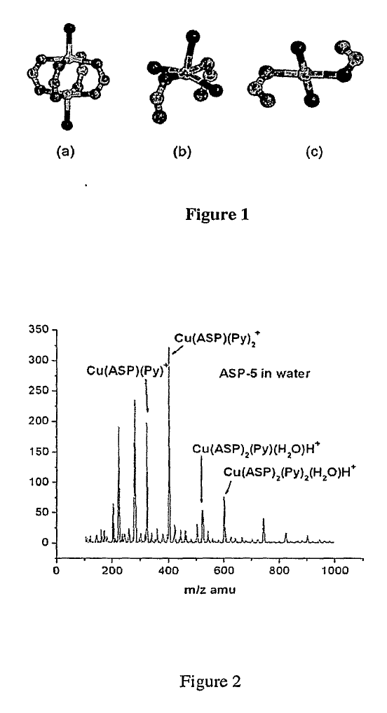

The foregoing data from the preparation and characterization of the 28 exemplary complexes of Example 1 demonstrates that lipophilicity and solubility of drug-metal complexes can be tuned by varying neutral ancillary ligands. The coordination chromophore of these complexes is illustrated in FIG. 1. Tables 2-5 list their respective solubilities in water (S_{w}), octanol-saturated water (S_{ow}), and water-saturated octanol (S_{wo}) at 25° C. The partition coefficient (logP) and solubility ratio logSR (log(S_{wo}/S_{ow})), for each compound have also been listed in Tables S1-S4. logP and logSR are commonly used as a suitable estimate of lipophilicity.⁶ The additive-constitutive character of partition coefficients within a congeneric series of compounds prepared from a parent organic drug is well established.⁷ More specifically, inductive effect, resonance effect and hydrogen bond are important factors that affect lipophilicity.⁸ These results demonstrate that the methods and compositions disclosed herein can be employed in systematic effort toward understanding in vitro quantitative structure-activity relationships (QSAR) for metal-drug complexes.

A. Stability of Mixed-Ligand Cu(II) Complexes in Solution.

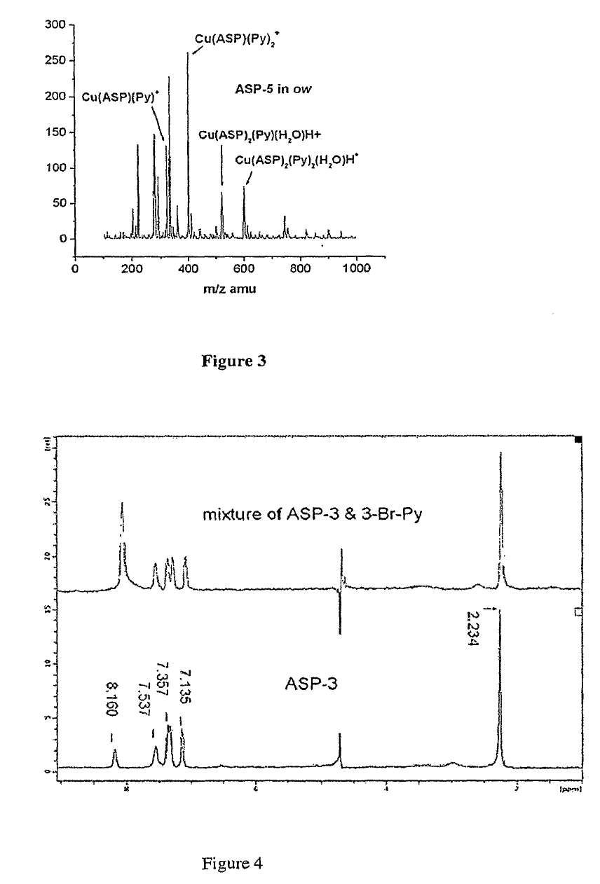

Previous thermodynamic results for associations of N-donor ligand and transition metal were used to infer the quality of the metal-ligand bonds and the stability of coordination species in solution. Gibbs free energy vales for the association of copper and N donor ligand (M²⁺+L=ML²⁺) were reported to be around −25˜−46 KJ/mol⁹, which suggest the association of transition metal and ligand are energy-favored. See FIGS. 2 and 3.

The electrospray ionization mass spectra of ASP-5 in water and octanol saturated water are shown in FIGS. 2 & 3 respectively. The spectra show that ASP-5 exists in aqueous solution as a monomer (Cu(ASP)₂(Py)₂(H₂O)H⁺, m/z 598.7; Cu(ASP)₂(Py)(H₂O)H⁺, m/z 519.7; Cu(ASP)(Py)₂⁺, m/z 399.8; Cu(ASP)(Py)⁺, m/z 320.8). The fragmentation of ASP-5 should result from the electrospray ionization.¹⁰ These masses indicate the formation of a monohydrate species of ASP-5, Cu(ASP)₂(AL)₂(H₂O). The formation of Cu(ASP)₂(AL)₂(H₂O) is also observed by us from the ESI-MS of ASP-7 and ASP-8 in aqueous solution. Indeed, for mononuclear Cu(II) species, the coordination of anionic API and neutral AL to copper in solution has been confirmed by the mass spectra.

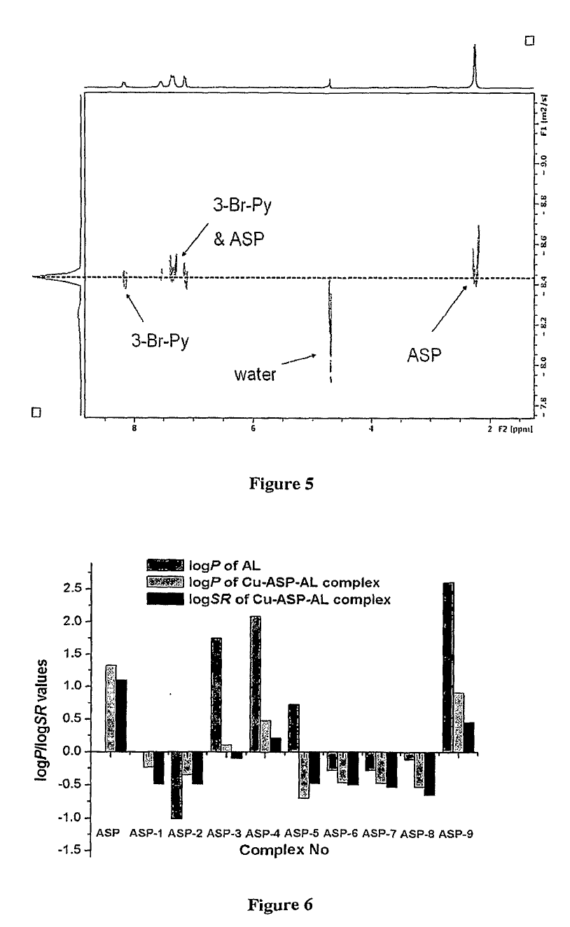

Since structures of binuclear Cu(II) complexes are analogous, as a model, ASP-3 was investigated by using NMR diffusion measurement for stability studies. Diffusion-Ordered Spectroscopy POSY) methods¹¹ are based on pulsed-field gradient spin-echo NMR experiments. In particular, DOSY is effective to analyze intermediate and to discriminate the different species in solution.¹² FIG. 4 shows the ¹H NMR spectra obtained for ASP-3 and the mixture after a small amount of 3-Br-Py has been added. The spectra shows a complicated pattern because of the existence of paramagnetic Cu(II) in solution.¹³ The sharp peak at 2.234 ppm is due to the aspirin methyl groups. The small peak on the left at 8.160 ppm is assigned to one proton of 3-Br-Py by comparing the ¹H NMR spectra of ASP-3 and ASP-3/3-Br-Py mixture. Peaks in the range of 7-8 ppm are due to the peak overstocking of aspirin and 3-Br-Py aromatic protons. A DOSY experiment was performed on a solution of ASP-3 in D₂O, and the results are shown in FIG. 5. The resulting ¹H NMR DOSY spectrum nicely suggests that aspirin anion and 3-Br-Py belongs to one species in water based on the diffusion coefficient. The diffusion coefficient for ASP-3 was 3.819×10⁻⁹ m²/s. This result indicates the homogeneity of ASP-3 in solution. Although no further determination of the configuration of ASP-3 in solution was undertaken in this study, it is clear from the DOSY spectrum the dissociation of mixed ligand Cu species in aqueous phase is insignificant.

B. Anionic APF with neutral ALF. Compounds containing non-steroidal anti-inflammatory drugs (NSAIDs) as ligands in copper(IT) complexes are known¹⁴; indeed, they have been shown to exhibit both enhanced efficacy and reduced side-effects.¹⁵ Specifically, aspirin (ASP)/salsalate (SAS) and a representative group of ancillary ligands were selected to form mixed ligand copper(II) coordination species, respectively. The partition coefficient (logP) and solubility ratio (logSR) for each compound for these two series have been shown graphically in FIGS. 6 & 7, respectively and set forth in Tables 2 and 3 below.

TABLE 2
Solubilities, Solubility Ratios, Partition Coefficients of
Aspirin and Complexes ASP-1 Through ASP-9 at 25° C.

| | | S_{w} | S_{ow} | S_{wo} | logSR | logP of |
|---|---|---|---|---|---|---|
| | logP of AL^{a} | (mg/ml) | (mg/ml) | (mg/ml) | (S_{wo}/S_{ow}) | complex |
| Aspirin(ASP) | / | 4.600^{b} | 4.295(10) | 54.16(94) | 1.101 | 1.321(25) |
| ASP-1 | / | 3.414(25) | 2.981(3) | 0.9767(36) | −0.4847 | −0.2371(267) |
| ASP-2 | −1.010(277) | 3.092(33) | 4.493(4) | 1.463(26) | −0.4873 | −0.3484(166) |
| ASP-3 | 1.751(286) | 1.564(3) | 1.566(5) | 1.256(15) | −0.09583 | 0.1066(58) |
| ASP-4 | 2.084(195) | 2.729(1) | 3.342(3) | 5.460(23) | 0.2133 | 0.4772(140) |
| ASP-5 | 0.726(178) | 3.425(21) | 3.629(24) | 1.210(47) | −0.4770 | −0.7001(111) |
| ASP-6 | −0.282(289) | 9.315(10) | 5.767(7) | 1.832(17) | −0.4980 | −0.4647(274) |
| ASP-7 | −0.282(289) | 9.259(5) | 5.654(20) | 1.673(9) | −0.5333 | −0.4723(272) |
| ASP-8 | −0.110(237) | 4.417(50) | 3.361(29) | 0.7541(97) | −0.6490 | −0.5257(136) |
| ASP-9 | 2.600(244) | 0.5415(7) | 0.4085(17) | 1.150(20) | 0.4495 | 0.9106(312) |

^{a}logP of ancillary ligands were calculated using Advanced Chemistry Development (ACD/Labs) Software V8.14 for Solaris.
^{b}from reference Koch, P. A.; Schultz, C. A.; Wills, R. J.; Hallquist, S. L.; Welling, P. G. Influence of food and fluid ingestion on aspirin bioavailability. J. Pharm. Sci. 1978, 67, 1533-1535.

TABLE 3
Solubilities, Solubility Ratios, Partition Coefficients of
Salsalate and Complexes SAS-1 Through SAS-9 at 25° C.

| | | S_{w} | S_{ow} | S_{wo} | logSR | logP of |
|---|---|---|---|---|---|---|
| | logP of AL^{a} | (mg/ml) | (mg/ml) | (mg/ml) | (S_{wo}/S_{ow}) | complex |
| Salsalate(SAS) | / | 0.59^{a} | 0.59^{b} | 87.83(38) | 2.176 | 3.045(353)^{a} |
| SAS-1 | / | 0.2791(6) | 0.2962(10) | 3.200(5) | 1.033 | 0.06927(1394) |
| SAS-2 | −0.131(371) | 0.3405(20) | 0.3227(6) | 0.6516(100) | 0.3051 | 0.05420(695) |
| SAS-3 | 1.569(214) | 0.1239(1) | 0.1259(4) | 0.7034(61) | 0.7472 | 0.06467(1468) |
| SAS-4 | 2.715(199) | 0.0134(3) | 0.0153(3) | 0.3314(22) | 1.336 | 0.7481(870) |
| SAS-5 | 2.590(242) | 0.0303(3) | 0.0315(2) | 1.236(18) | 1.593 | 1.231(53) |
| SAS-6 | 0.726(178) | 0.4065(8) | 0.3291(8) | 1.918(5) | 0.7655 | 0.03218(199) |
| SAS-7 | −0.282(289) | 0.3143(9) | 0.2890(10) | 0.8344(165) | 0.4241 | 0.03802(967) |
| SAS-8 | 1.186(185) | 0.2301(18) | 0.2174(10) | 1.932(23) | 0.9488 | 0.01407(1658) |
| SAS-9 | 1.718(185) | 0.1143(2) | 0.1104(8) | 1.438(9) | 1.115 | 0.02089(576) |

^{a}logP of ancillary ligands/salsalate and water solubility of salsalate were calculated using Advanced Chemistry Development (ACD/Labs) Software V8.14 for Solaris.
^{b}S_{ow} and S_{wo} of salsalate were supposed to be equal.

For the binuclear Cu(II)-aspirinate species, logP and logSR values were significantly increased via the introduction of both 3-Br-Py and quinoline. The order of logP/logSR values for the dicopper complexes, Cu₂(ASP)₄(AL)₂, reflects the order of logP values calculated for the ancillary ligands (FIG. 6), which is consistent with the additive-constitutive character observed for organic congeners. For the mononuclear Cu(II)-aspirinate species, a similar general tendency is observed. The exception being that, although the logP values of isonicotinamide and nicotinamide are smaller than Py, the SR values for complexes ASP-(6-8) are close to the SR value for ASP-5; moreover, the logP values are greater. This indicates that the lipophilicity of complexes ASP-(6-8) is greater than expected based on the logP value of the ancillary ligand. This is attributed to hydrogen bonding, which is consistent with the additive-constitutive character of hydrogen bonding functionalities in metal-free solid forms where it has been suggested that hydrogen bonding reduces the affinity for the aqueous phase and thus increases the relative lipophilicity.¹⁶ In comparing the binuclear and mononuclear Cu(II)-aspirinate species, the complexes that possess the dicopper chromophore are more lipophilic than the mononuclear complexes. ESI-MS of complexes ASP-(5, 6 & 8) suggest the monocopper complex exists as a hydrated species, Cu(ASP)₂(AL)₂(H₂O), in the aqueous phase. This presumably increases the aqueous solubility and thus decreases its relative lipophilicity.

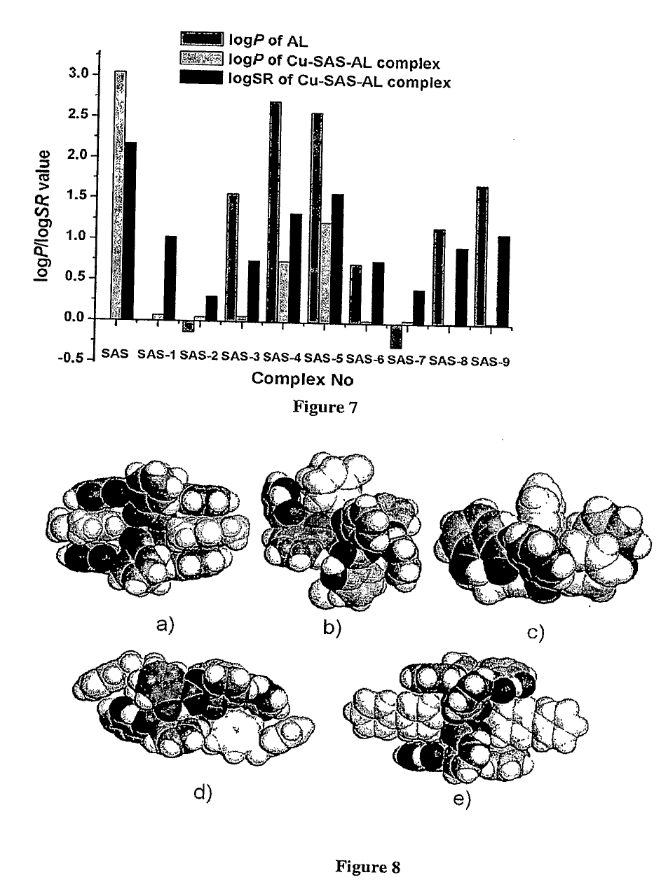

Within the Cu(II)-salsalate series, the order of logSR values for the Cu(II)-salsalate species with the same coordination chromophore reflects the order of logP values calculated for the ancillary ligand functionalities, which is also consistent with the additive-constitutive character observed for organic congeners. The exception being that, although the logP value of 4-Benzylpyridine is higher than 4-Phenylpyridine, the logSR/logP values of SAS-4 is smaller than SAS-5. This might be due to the uncertainty of calculated logP values of ancillary ligands. It's also worth noting that although the logP values calculated for the ancillary ligand functionalities vary in a wide rang (from −0.282 to 2.715), Table S2, only the logP values of SAS-4 and SAS-5 were significantly increased via the introduction of aromatic ancillary ligands functionalities, FIG. 7. This is attributed to size effects. Since the phenol groups in salsalate sterically shields the ancillary ligands, then aqueous interactions will decrease, thus the logP values of Cu-Salsalate-ALF species with a small ancillary ligand functionality do not change much. As exemplified in FIG. 8, the ancillary ligand functionalists were shielded by salsalate phenol rings, and notably ancillary ligands in SAS-2 & SAS-5 are sandwiched between two phenol rings. So even if 4-Benzylpyridine and 4-Phenylpyridine are sterically shielded by salsalate phenol rings somehow, only the logP values of corresponding complexes SAS-4 & SAS-5 were still significantly increased because of the large molecular size of ancillary ligands.

There is also some degree of predictability in observed aqueous solubility based on the logP of the ancillary ligand. Generally with the increase of logP value of ancillary ligand, aqueous solubilities of Cu-APF-ALF congeners with the same coordination chromophore decrease, as is shown in Table S1 & S2.

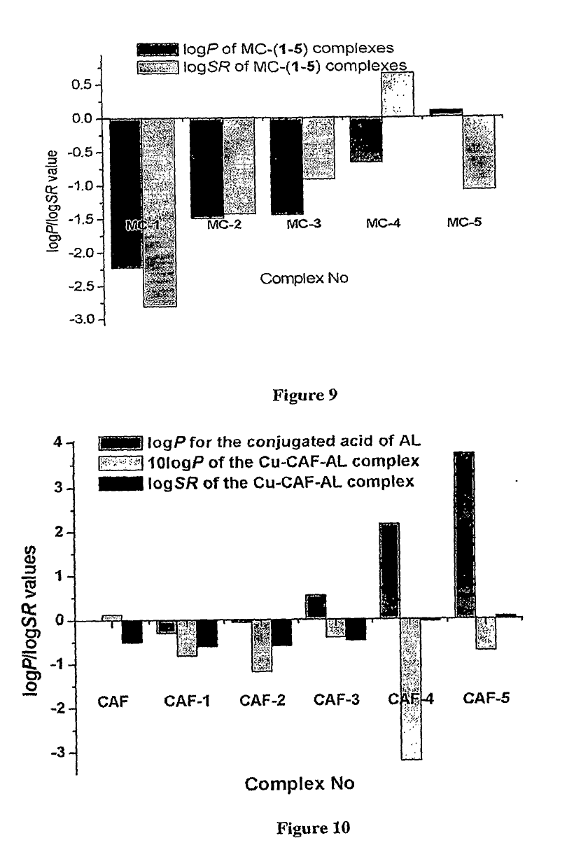

C. Mixed anionic ligand complexes. Dinuclear Cu(II) paddle-wheel complexes with four identical carboxylates are ubiquitous, and only rare examples of mixed carboxylate dinuclear Cu(II) complexes are known so far.¹⁷ Cu₂(Ac)₂(DMB)₂(H₂O)₂, Cu₂(DMB)₄(H₂O)₂ (HAc=acetic acid, H(DMB)=2,6-dimethoxy benzoic acid), Cu₂(Ac)₂(VA)₂(H₂O)₂, Cu₂(VA)₄(H₂O)₂ (H(VA)=vanillic acid) were prepared in water solution or formed under moist conditions. Then Cu₂(Ac)₄(H₂O)₂ (MC-1), Cu₂(Ac)₂(DMB)₂(H₂O)₂ (MC-2), Cu₂(DMB)₄(H₂O)₂ (MC-3), Cu₂(Ac)₂(VA)₂(H₂O)₂ (MC-4) and Cu₂(VA)₄(H₂O)₂ (MC-5) were investigated as proof of principle complexes, whereas neither 2,6-dimethoxy benzoic acid nor vanillic acid is an APF. Since the carboxylate ligand is “negatively” charged, we can only compare the lipophilicity of their corresponding conjugated acid. The calculated logP values of acetic acid, 2,6-dimethoxy benzoic acid and vanillic acid are −0.285(184), 0.975(256) and 1.334(245), respectively. As shown in FIG. 9, The logP/logSR values for MC-3 are significantly higher than those for MC-1, which indicates MC-3 is more lipophilic than MC-1. And the logP/logSR values for the mixed carboxylate complex, MC-2, is between logP/logSR values for MC-1 and MC-3 (Table S3), which is also qualitatively consistent with the additive-constitutive character observed for organic congeners. Similar tendency has also been observed among MC-1, 4 & 5. The only exception is that the logSR value of MC-5 is lower than the logSR value of MC-4, which is due to the low octanol solubility for MC-5, Table S3. Presumably the low S_{ow} value for MC-5 was resulted from the relative high lattice energy. Particularly, the logP value for MC-2 is slightly lower than the logP value for MC-3. This is due to the small size of acetate anion, which can not effectively change the lipophilicity by ligand substitution.

TABLE 4
Solubilities, Solubility Ratios, Partition Coefficients
of Complexes MC-1 Through MC-5 at 25° C.

| | S_{w} | S_{ow} | S_{wo} | logSR | logP of |
|---|---|---|---|---|---|
| | (mg/ml) | (mg/ml) | (mg/ml) | (S_{wo}/S_{ow}) | complex |
| MC-1 | 72^{a} | 74.47(136) | 0.1161(438) | −2.807 | −2.222(553) |
| MC-2 | 15.82(11) | 15.64(14) | 0.5841(103) | −1.428 | −1.496(15) |
| MC-3 | 33.61(21) | 31.97(19) | 3.872(125) | −0.9168 | −1.442(27) |
| MC-4 | 0.1257(12) | 0.1257(12) | 0.1257(12) | 0.6573 | −0.6626(353) |
| MC-5 | 0.1257(12) | 0.1257(12) | 0.1257(12) | −1.081 | 0.0928(3389) |

^{a}S_{w} of Cu₂(Ac)₄(H₂O)₂ is from MSDS.

D. Neutral APF with anionic anionic APF. Caffeine (CAF) is one imidazole type purine alkaloids with interesting pharmacological properties. Copper(II)-carboxylate-(imidazole type ALF) mixed ligand species have also been found to have a variety of pharmacological effects such as anticancer¹⁸, superoxide dismutase¹⁹, and catecholase mimetic activities²⁰. Table S4 below lists solubility, partition coefficient and solubility ratio for each prepared Copper(II)-carboxylate-caffeine complex. As mentioned above, since the carboxylate ligand is “negatively” charged, the calculated logP value for the corresponding conjugated acid was used as a quantitative parameter.

TABLE 5
Solubilities, Solubility Ratios and Partition Coefficients of
Caffeine and Complexes CAF-1 Through CAF-5 at 25° C.

| | logP of | S_{w} | S_{ow} | S_{wo} | logSR | logP of |
|---|---|---|---|---|---|---|
| | H(AL)^{a} | (mg/ml) | (mg/ml) | (mg/ml) | (S_{wo}/S_{ow}) | complex |
| caffeine(CAF) | / | 24.5(3) | 23.5(2) | 7.35(23) | −0.5045 | 0.0121(855) |
| CAF-1 | −0.285(184) | 39.94(5) | 45.69(6) | 11.42(8) | −0.6022 | −0.08119(2402) |
| CAF-2 | −0.047(237) | 56.67(124) | 54.10(33) | 13.86(27) | −0.5914 | −0.1177(57) |
| CAF-3 | 0.542(290) | 26.42(16) | 31.48(9) | 10.58(19) | −0.4735 | −0.03997(751) |
| CAF-4 | 2.163(325) | 5.024(68) | 3.262(13) | 2.980(63) | −0.03924 | −0.3210(1356) |
| CAF-5 | 3.722(227) | 5.160(9) | 5.584(2) | 6.547(231) | 0.06893 | −0.07137(4156) |

^{a}logP of the conjugated acid for ancillary ligands were calculated using Advanced Chemistry Development (ACD/Labs) Software V8.14 for Solaris.

Within this series of compounds, the order of logSR values follows the order of logP values for the conjugated acid of the ancillary ligands, FIG. 10. Complexes CAF-(1-5) fall into two categories based on the observed logP values. CAF-(1, 2 & 3): The logP values for complexes CAF-(1, 2 & 3) are close to the logP value of caffeine. The logP for CAF-3 is the highest among these three species because of the relative higher logP value of dichloro acetic acid than acetic acid and monochloro acetic acid. Caf-(4 & 5): The order of observed logP values also follows the order of logP values for the conjugated acid of the ancillary ligands. However, even if the logP values of 2-iodine-benzoic acid and ibuprofen are higher than acetic acid/monochloro acetic acid/dichloro acetic acid, the logP values for CAF-(4 & 5) are lower than logP values for CAF-(1, 2 & 3). Presumably this is due to the small size of the ancillary ligands in CAF-(1, 2 & 3), which also explain why the logP value for CAF-(1, 2 & 3) are closer to caffeine's logP value.

### CONCLUSION

Electrospray mass spectrometry and DOSY NMR data indicate that the mixed ligand Cu(II) species are stable in the aqueous phase, which ensure the validity of data measured and disclosed herein. The introduction of ancillary ligand functionalities can form a homologous series of metal-drug complexes with variable lipophilicity and solubility. The solubility and lipophilicity of a drug are key parameters that influence its ADMET properties.²¹ In particular, the relative potency of the drug is related to its lipophilicity by a certain mathematical representation.²² So, the introduction of ancillary ligand functionalities may also be expected to modify the potency and efficacy of the parent metal-drug complexes.

Our data suggest that the formation of mixed ligand coordination species can be developed as a complementary technique to pharmaceutical co-crystals for fine tuning critical ADMET parameters without altering the parent drug compound via organic transformations. The most significant finding is that we have demonstrated herein that there is predictability in the observed lipophilicity and solubilities based on the logP of the ancillary ligand functionality. More specifically, additive-constitutive strategies to tune the lipophilicity of organic molecules can be qualitatively extended to metal-drug complexes. The nature of the chromophore, hydrogen bonds and relative size of the ligands are also important factors that affect the lipophilicity of metal-drug complexes.

The use of metals for medicinal purpose is not new. Extensive studies of metals in medicine including metal based drugs and metal based diagnostic agents have been carried out since last century.²³ However, we are unaware of a developed, general, and rational approach to the modification of metal-drug complexes other than that described herein. Some reported results have indicated the efficacy of metal-drug complexes can be improved by the introduction of a proper ancillary ligand.²⁴ There are also other advantages of the introduction of ancillary ligand functionalities over altering the patent drug compound via organic transformations: easy preparation, integrity of APF, lower cost. One of the anticipated concerns regarding the use of metal-based coordination species may be the accumulation and toxicity of the metal. Since many transition metals are considered dietary micronutrients (i.e. Cr, Mn, Fe, Cu, Zn), the daily intake of certain transition metals under the Tolerable Upper Intake level is supposed to pose no risk of adverse health effects for almost all individuals in the general population.²⁵ We have therefore initially targeted these metals in our studies. In conclusion, we demonstrate that complexes that incorporate TMFs coordinated to APFs and property-directing ALFs offer many opportunities for the development and commercialization of new or existing drugs. More generally, supramolecular synthesis—which includes metal complexes and co-crystals—is a valuable tool for fine-tuning critical physical properties, improving processability. This appears to be a novel approach to affect the lipophilicity of ancillary metal functionalities. The demonstration that the lipophilicity of prototypical APFs may be greatly affected without direct, chemical modification will provide an ability to alter other pharmacodynamic and pharmacokinetic properties such as bulk solubility, bioavailability, and biological action. The incorporation of the ALF is critical in the ability to afford advantageous physical properties, as it is shown that coordination of the APF to a TMF without the ALF offers little advantage or flexibility over the APF itself.

All of the compositions and methods disclosed and claimed herein can be made and executed without undue experimentation in light of the disclosure. Although the compositions and methods of the invention have been described in terms of preferred embodiments, it will be apparent to those having ordinary skill in the art that variation may be made to the compositions and methods without departing from the concept, spirit, and scope of the inventions. For, example, certain agents and compositions that are chemically related may be substituted for the agents described herein if the same or similar results would be achieved. All such similar substitutes and modifications apparent to those skilled in the art are deemed to be within the spirit, scope, and concept of the invention.

All publications, patent applications, patents and other documents cited herein are incorporated by reference in their entirety. In case of conflict, this specification including the definitions will control. In addition, the material, methods, and examples are illustrative only and not intended to be limiting.

### REFERENCES

(1) Lehn, J. M. Supramolecular Chemistry—Scope and perspectives molecules, supermolecules, and molecular devices. Angew. Chem. Int. Ed. 1988, 27, 89-112.; Moulton, B,; Zaworotko, M. J. From molecules to crystal engineering. Supramolecular isomerism and polymorphism in network solids. Chem. Rev. 2001, 101, 1629-1658.; Aakeroey, C. B.; Desper, J.; Smith, M.; Urbina, J. F. [(Benzimidazol-1-yl)methyl]benzamides as bifunctional reagents for reliable inorganic-organic supramolecular synthesis. Dalton Trans. 2005, 2462-2470.
(2) Smith, D. K. A Supramolecular Approach to Medicinal Chemistry: Medicine Beyond the Molecule. J. Chem. Edu. 2005, 82, 393-400.
(3) Vishweshwar, P.; McMahon, J. A.; Bis, J. A.; Zaworotko, M. J. Pharmaceutical co-crystals. J. Pharm. Sd. 2006, 95, 499-516.; Trask, A. V.; Samuel Mother Well, W. D.; Jones, W. Pharmaceutical Cocrystallization: Engineering a Remedy for Caffeine Hydration. Cryst. Growth Design. 2005, 5, 1013-1021.; Reddy, L. S.; Babu, N. J.; Nangia, A. Carboxamide-pyridine N-oxide heterosynthon for crystal engineering and pharmaceutical cocrystals, Chem. Commun., 2006, 1369-1371.; Almarsson, O.; Zaworotko, M. J. Crystal engineering of the composition of pharmaceutical phases. Do pharmaceutical co-crystals represent a new path to improved medicines? Chem. Commun., 2004, 1889-1896.
(4) Weder. Copper Complexes of Non-steroidal Anti-inflammatory Drugs: An Opportunity Yet to Be Realized. Coordination Chemistry Reviews 2002, 232, 95-126
(5) Ma, Z.; Moulton, B. Mixed-ligand coordination species: a promising approach for “second-generation” drug development. Cryst. Grow. Design. In press.
(6) Yalkowsky, S. H.; Valvani, S. C.; Roseman, T. J. Solubility and partitioning VI: octanol solubility and octanol-water partition coefficients. J. Pharm. Sci. 1983, 72, 866-870.; Pinsuwan, S.; Li, A.; Yalkowsky, S. H. Correlation of Octanol/Water Solubility Ratios and Partition Coefficients. J. Chem. Eng. Data. 1995, 40, 623-626.; Jozan, M.; Takacs-Novak, K. Determination of solubilities in water and 1-octanol of nitrogen-bridgehead heterocyclic compounds. Int. J. Pharm. 1997, 159, 233-242.
(7) Fujita, T.; Iwasa, J.; Hansch, C. A New Substituent Constant, π, Derived from Partition Coefficients. J. Am. Chem. Soc. 1964, 86, 5175-5180.
(8) Leo, A.; Hansch, C.; Elkins, D. Partition coefficients and their uses. Chem. Rev. 1971, 71, 525-616.
(9) Aruga, R. Thermodynamics of Complex Formation of 1-Methylimidazole with Cobalt(II), Nickel(II) and Zinc(II) Cations in Aqueous Solution. Trans. Met. Chem. 1983, 8, 56-58.; Arena, G.; Cali, R.; Rizzarelhi, E.; Sammartano, S.; Barbucci, R.; Campbell, M. J. M. Thermodynamic and Spectroscopic Properties of Mixed Complexes in Aqueous Solution. Copper(II) Complexes of 2,2′-Bipyridyl and Cycloalkane-1,1-dicarboxylic Acids. J.C.S. Dalton. 1977, 581-585.
(10) Jones, E. C.; Taylor, P. J.; McEwan, A. G.; Hanson, G. R. Spectroscopic Characterization of Copper(II) Binding to the Immunosuppressive Drug Mycophenolic Acid. J. Am. Chem. Soc. 2006, 128, 9378-9386.; van den Brenk, A. L.; Byriel, K. A.; Fairlie, D. P.; Gahan, L. R.; Hanson, G. R.; Hawkins, C. J.; Jones, A.; Kennard, C. H. L.; Moubaraki, B.; Murray, K. S. Crystal Structure and Electrospray Ionization Mass Spectrometry, Electron Paramagnetic Resonance, and Magnetic Susceptibility Study of [Cu2(ascidH2)(1,2-.mu.-CO3)(H2O)2].cntdot.2H2O, the Bis(copper(II)) Complex of Ascidiacyclamide (ascidH4), a Cyclic Peptide Isolated from the Ascidian Lissoclinum patella. 1994, 33, 3549-3557.
(11) Morris, K. F.; Johnson, C. S. Diffusion-ordered two-dimensional nuclear magnetic resonance spectroscopy. J. Am. Chem. Soc. 1992, 114, 3139-3141.; Morris, K. F.; Johnson, C. S. Resolution of discrete and continuous molecular size distributions by means of diffusion-ordered 2D NMR spectroscopy. J. Am. Chem. Soc. 1993, 115, 4291-4299.
(12) Allouche, L.; Marquis, A.; Lehn, J. Discrimination of Metallosupramolecular Architectures in Solution by Using Diffusion Ordered Spectroscopy (DOSY) Experiments: Double-Stranded Helicates of Different Lengths. Chem. Eur. J. 2006, 12, 7520-7525.; Claridge, T. D. W. High-Resolution Nmr Techniques in Organic Chemistry. Elsevier: New York, 1999; pp 187-200.
(13) Ramadan, S.; Hambley, T. W.; Kennedy, B. J.; Lay, P. A. NMR Spectroscopic Characterization of Copper(I) and Zinc(II) Complexes of Indomethacin. Inorg. Chem. 2004, 43, 2943-2946.
(14) Sorenson, J. R. J. Copper chelates as possible active forms of the antiarthritic agents. J. Med. Chem. 1976, 19, 135-148.
(15) Milanino, R.; Rains ford, K. D.; Velo, G. P. Copper and Zinc in Inflammation; Kluwer: Dordrecht, 1989.; Weder, J. E.; Dillon, C. T.; Hambley, T. W.; Kennedy, B. J.; Lay, P. A.; Biffin, J. R.; Regtop, H. L.; Davies, N. M. Copper complexes of non-steroidal ant-inflammatory drugs: an opportunity yet to be realized. Coord. Chem. Rev. 2002, 232, 95-126.
(16) Silverman, R. B. The Organic Chemistry of Drag Design and Drug Action; Academic Press: Longdon, 1992; pp 30-31.; Charton, M.; Charton, B. I. Hydrogen bonding contribution to lipophilicity parameters. Hydrogen acceptor and hydrogen acceptor donor parameters. Collet. Czech. Chem. Comm. 2004, 69, 2147-2173.
(17) Maspoch, D.; Ruiz-molina, D.; Wurst, K.; Rovira, C.; Veciana, J. A very bulky carboxylic perchlorotriphenylmethyl radical as a novel ligand for transition metal complexes. A new spin frustrated metal system. Chem. Commun. 2002, 2958-2959.; Vives, G.; Mason, S. A.; Prince, P. D.; Junk, P. C.; Steed, J. W. Intramolecular versus Intermolecular Hydrogen Bonding of Coordinated Acetate to Organic Acids: A Neutron, X-ray, and Database Study. Cyst. Grow. Design. 2003, 3, 699-704.; Strinnaerre, L.; Micera, G.; Piu, P.; Cariati, F.; Clani, G. Synthesis, Spectral Properties, and Crystal and Molecular Structure of Tetrakis(μ-2,6-dimethoxybenzoato)diaquadicopper(II) and of Bis(μ-2,6-dimethoxybenzoato)bis(μ-acetato)diaquadicopper(II), a Case of a Dimeric Copper(II) Carboxylate Complex with Mixed Bridges. Inorg. Chem. 1985, 24, 2297-2300.; Banares, M. A.; Dauphin, L.; Lei, X.; Cen, W.; Shang, M.; Wolf, E. E.; Fehlner, T. P. Effect of Precursor Core Structure on the Hydrogenation of 1,3-Butadiene Catalyzed by Cluster-Derived Model Catalysts. Chem. Mater. 1995, 7, 553-561.; Kozlevcar, B.; Odlazek, D.; Golobic, A.; Pevec, A.; Strauch, P.; Segedin, P. Complexes with lignin model compound acid. Two different carboxylate ligands in the same dinuclear tetracarboxylate complex [Cu₂(C₈H₇O₄)₂(O₂CCH₃)₂(CH₃OH)₂]. Polyhedron, 2006, 25, 1161-1166.
(18) Tamua, H.; Imai, H.; Ksuwahara, J.; Suguira, Y. A new antitumor complex: bis(acetato)bis(imidazole)copper(II). J. Am. Chem. Soc. 1987, 109, 6870-6871.
(19) Bhirud, R. G.; Srivastava, T. S. Superoxide dismutase activity of Cu(II)₂(aspirinate)₄ and its adducts with nitrogen and oxygen donors. Inorg. Chim. Adca. 1990, 173, 121-125.
(20) Abuhijleh, A. L.; Woods, C.; Bogas, E.; LeGuenniou, G. Synthesis, characterization and catecholase-mimetic activity of mononuclear copper(II) aspirinate complexes. Inorg. Chim. Acta. 1992, 195, 67-71.
(21) Lipinski, C. A.; Lombardo, F.; Dominy, B. W.; Feeney, P. J. Experimental and computational approaches to estimate solubility and permeability in drug discovery and development settings. Adv. Drug Del. Rev. 1997, 23, 3-25.
(22) Hansch, C.; Steward, A. R.; Anderson, S. M.; Bentley, D. Parabolic dependence of drug action upon lipophilic character as revealed by a study of hypnotics. J. Med. Chem. 1968, 11, 1-11.
(23) Guo, Z.; Sadler, P. J. Metals in Medicine. Angew. Chem. Int. Ed. 1999, 38, 1512-1531.
(24) van Kralingen, C. G.; Reedijk, J.; Spek, A. L. Synthesis and characterization of antitumor-active platinum 2,2-dimethyl-1,3-diaminopropane compounds. Crystal and molecular structure of (malonato) (2,2-dimethyl-1,3-diaminopropane)platinum(II). Inorg. Chem. 1980, 19, 1481-1485.
(25) Food & Nutrition Board Standing Committee on the Scientific Evaluation of Dietary Reference. Dietary Reference Intakes: For Vitamin A, Vitamin K, Arsenic, Boron, Chromium, Copper, Iodine, Iron, Manganese, Molybdenum, Nickel, Silicon, Vanadium, and Zinc. Natl Academy Press: 2002; pp 7-8.

We claim:

1. A neutral, multi-functionality assembly of an active pharmaceutical composition, said composition comprising a transition metal functionality (TMF), an active medicinal functionality (AMF), and an ancillary ligand functionality (ALF), at least one of the AMF and the ALF having one or more anionic elements forming one or more covalent bonds with the TMF such that the total molecular chare of the composition is neutral.

2. The composition of claim 1, wherein the TMF comprises a cation having a charge of +1, +2, +3, or +4.

3. The composition of claim 2, wherein the TMF comprises a cation having a charge of +2 or +3.

4. The composition of claim 3, wherein the TMF is selected from the group consisting of Iron (Fe), Zinc (Zn), Copper (Cu), Manganese (Mn), Chromium (Cr), Molybdenum (Mo), Cobalt (Co), Nickel (Ni), Vanadium (V), Silver (Ag), Platinum (Pt), Gold (Au), Scandium (Sc), Titanium (Ti), Yttrium (Y), Zirconium (Zr), Niobium (Nb), Technetium (Tc), Ruthenium (Ru), Rhodium (Rh), palladium (Pd), Cadmium (Cd), Hafnium (Hf), Tantalum (Ta), Tungsten (W), Rhenium (Re), Osmium (Os), Iridium (Ir), Mercury (Hg), and the inner transition metal members of the lanthanide and actinide series.

5. The composition of claim 4, wherein the TMF is a bioTMF.

6. The composition of claim 5, wherein the bioTMF is Cu.

7. The composition of claim 1, wherein the APF contains one or more moieties to which the other functionalities are covalently bound, said moieties being selected from the group consisting of acids, amides, aliphatic nitrogen bases, unsaturated aromatic nitrogen bases, pyridines, imidazoles, amines, alcohols, halogens, sulfones, nitro groups, S-heterocycles, saturated or unsaturated N-heterocycles, O-heterocycles, ethers thioethers, thiols, esters, thioesters, thioketones, epoxides, acetonates, nitriles, oximes and organohalides.

8. The composition of claim 1, wherein the ALF is a solvent, another pharmaceutical molecule, or a GRAS compound.

9. A neutral, multi-functionality assembly of an active pharmaceutical composition, said composition comprising a TMF selected from the group consisting of Iron (Fe), Zinc (Zn), Copper (Cu), and Manganese (Mn); an AMF containing one or more moieties selected from the group consisting of acids, amides, aliphatic nitrogen bases, unsaturated aromatic nitrogen bases, pyridines, imidazoles, amines, alcohols, halogens, sulfones, nitro groups, S-heterocycles, saturated or unsaturated N-heterocycles, O-heterocycles, ethers, thioethers, thiols, esters, thioesters, thioketones, epoxides, acetonates, nitrites, oximes, and organohalides; and an ALF, at least one of the AMF or the ALF having one or more anionic elements forming one or more covalent bonds with the TMF such that the total molecular charge of the composition is neutral.

10. The composition of claim 9, wherein the ALF is a solvent, another pharmaceutical molecule, or a GRAS compound.

11. A method of designing a neutral multi-functionality assembly of an active pharmaceutical composition composed of an AMF, a cationic TMF, and an ALF, the method comprising:
(a) selecting an AMF having a moiety capable of covalently binding the cationic TMF;
(b) identifying an ALF having a moiety capable of covalently binding the cationic TMF; and
(c) arranging the functionalities such that the total molecular charge of the assembly composed of the combined functionalities is neutral.

12. The method of claim 11, wherein the TMF is a bioTMF.

13. The method of claim 12, wherein the bioTMF is Cu.

14. The method of claim 11, wherein the ALF is a solvent, another pharmaceutical molecule, or a GRAS compound.

15. The method of claim 11, wherein the AMF contains one or more moieties selected from the group consisting of acids, amides, aliphatic nitrogen bases, unsaturated aromatic nitrogen bases, pyridines, imidazoles, amines, alcohols, halogens, sulfones, nitro groups, S-heterocycles, saturated or unsaturated N-heterocycles, O-heterocycles, ethers, thioethers, thiols, esters, thioesters, thioketones, epoxides, acetonates, nitrites, oximes, and organohalides.

16. A method of designing a neutral multi-functionality assembly of an active pharmaceutical composition composed of an AMF, a cationic TMF, and an ALF, the method comprising:
(a) selecting an AMF having one or more moieties capable of covalently binding the cationic TMF, the one or more moieties being selected from the group consisting of acids, amides, aliphatic nitrogen bases, unsaturated aromatic nitrogen bases, pyridines, imidazoles, amines, alcohols, halogens, sulfones, nitro groups, S-heterocycles, saturated or unsaturated N-heterocycles, O-heterocycles, ethers, thioethers, thiols, esters, thioesters, thioketones, epoxides, acetonates, nitriles, oximes, and organohalides;
(b) identifying an ALF having a moiety capable of covalently binding the cationic TMF, the ALF being a solvent, another pharmaceutical molecule, or a GRAS compound; and
(c) arranging the functionalities such that the total molecular charge of the assembly composed of the combined functionalities is neutral.

17. The method of claim 16, wherein the TMF is a bioTMF.

18. The method of claim 17, wherein the bioTMF is Cu.
